(19) United States
(12) Patent Application Publication
(10) Pub. No.: US 2024/0390067 A1
(43) Pub. Date: Nov. 28, 2024

(54) METHOD OF RECONSTRUCTING CARTILAGE OF DISTAL FEMUR

(71) Applicants: Kyoung Tak KANG, Seoul (KR); Ji Hoon NAM, Seoul (KR); TJC Life Co., Ltd., Seoul (KR)

(72) Inventors: Kyoung Tak KANG, Seoul (KR); Ji Hoon NAM, Seoul (KR); Yong Gon KOH, Seoul (KR)

(73) Assignees: Kyoung Tak KANG, Seoul (KR); Ji Hoon NAM, Seoul (KR); TJC Life Co., Ltd., Seoul (KR)

(21) Appl. No.: 18/346,322

(22) Filed: Jul. 3, 2023

(30) Foreign Application Priority Data
May 22, 2023 (KR) 10-2023-0065875

(51) Int. Cl. A61B 34/10; G06T 7/00

(52) U.S. Cl. CPC A61B 34/10; G06T 7/0012; A61B 2034/105; A61B 2034/107

(57) ABSTRACT

Proposed is a method of reconstructing a damaged cartilage of a distal femur to a pre-damaged state for the design of a patient-specific artificial knee joint. The method includes generating a distal femur model (A) having only a bone without a cartilage from image data of a femur captured by a medical imaging device, generating a distal femur model (B) having a damaged cartilage from the image data, extracting a bone cross section for each region in each of the model (A) and the model (B), obtaining a distance between an articular surface of the model (A) and a cartilage surface of the model (B) by using a bone cross section of the model (A) and a bone cross section of the model (B) which are extracted from the same region, and generating a distal femur model (C) with a cartilage reconstructed according to a reconstruction line.

### CROSS REFERENCE TO RELATED APPLICATION

The present application claims priority to Korean Patent Application No. 10-2023-0065875, filed May 22, 2023, the entire contents of which are incorporated herein for all purposes by this reference.

### BACKGROUND OF THE INVENTION

### Field of the Invention

The present disclosure relates generally to a method of reconstructing cartilage of a distal femur. More particularly, the present disclosure relates to a method of reconstructing cartilage of a distal femur, in which the damaged cartilage of a distal femur is reconstructed to a pre-damaged state thereof by using a 3D drawing program so as to design a patient-specific artificial joint.

### Description of the Related Art

A knee joint refers to a joint made up of three bones adjacent to each other that surround the knee: the femur (also called “a thigh bone”), the tibia (also called “a shin bone”), and the patella (also called “a kneecap”). The knee joint is present in both left and right legs, and together with the hip joint (a coxa) is the most essential joint enabling the human body to walk upright.

The knee joint has a high frequency of use due to its nature, leaving a lot of room for damage to occur. In particular, due to abrasion or aging of a bone tissue, the knee joint may have deterioration or loss of a function thereof, and accordingly, due to gradual damage or degenerative change of cartilage, a bone and a ligament may be damaged, causing inflammation and pain. This is commonly referred to as degenerative arthritis.

As a method of treating degenerative arthritis, conservative treatments such as lifestyle improvement, drug treatment, and physical therapy are known. However, these treatment methods only slow down the progression of degenerative arthritis or temporarily relieve pain, but do not provide a fundamental solution. In particular, there are cases in which degenerative arthritis is extremely aggravated and difficult to treat with medication or physical therapy.

A total knee arthroplasty (TKA) is performed for a patient whose degenerative arthritis is extremely aggravated and pain continues despite drug or physical therapy that it is difficult for the patient to walk. The total knee arthroplasty (TKA) is a surgical procedure in which a damaged joint is removed from a patient with severely advanced degenerative arthritis and then an artificial joint made of special metal and plastic is inserted.

For reference, the artificial joint used in the total knee arthroplasty is generally composed of a femur element, a tibia element, and a bearing element. Here, the femur element is implanted in a location at which an articular surface of a severely worn femoral joint is resected, and the tibia element is implanted in a location at which the articular surface of the upper end of the tibia adjacent to the femoral joint is resected. The bearing element functions as a kind of cartilage between the femur element and the tibia element.

Among total knee arthroplasties, a surgical method that has recently been spotlighted is a patient-specific total knee arthroplasty. Since the patient-specific total knee arthroplasty is performed to preserve a patient's knee shape and biomechanics as much as possible, the patient-specific total knee arthroplasty has the advantage of being able to reproduce the motion of the knee joint after surgery close to a normal state thereof before surgery, and therefore, the satisfaction of patients who underwent the surgery is quite high.

In the patient-specific total knee arthroplasty, an artificial joint to replace a damaged joint is required to be designed according to a patient's knee shape and biomechanics. One of important factors considered in this design process is the thickness of the cartilage of the distal femur in a normal state. However, since the actual cartilage of the distal femur of a patient is already damaged, it is difficult to design an artificial joint customized for the patient in that state.

Accordingly, a conventional technology adopts a method in which a CT image of the distal femur is used to reconstruct the bone of the distal femur in the form of a 3D digital model by using a 3D drawing program, a virtual cartilage is generated by being offset by a predetermined distance from the articular surface of the reconstructed 3D bone model, and a joint line and a posterior line are generated from the generated virtual cartilage as illustrated in FIG. 1 so that the lines are considered to design an artificial joint.

However, in this method, the virtual cartilage is generated by being offset by a predetermined thickness (approximately 1 to 2 mm) from the articular surface of the 3D bone model without considering the thickness of an actual cartilage that is not damaged, and accordingly, the thickness of the generated virtual cartilage is smaller or greater than the thickness of the normal cartilage before damage. Therefore, there is a problem in that the artificial joint cannot be designed in an optimal form in which the artificial joint fits a patient's actual anatomical characteristics.

### Document of Related Art

(Patent Document) Korean Patent Application Publication No. 10-2022-0148364 (published on Nov. 7, 2022)

### SUMMARY OF THE INVENTION

Accordingly, the present disclosure has been made keeping in mind the above problems occurring in the related art, and the present disclosure is intended to propose a method of reconstructing cartilage of a distal femur, in which when reconstructing damaged cartilage of a distal femur to a pre-damaged state thereof by using a 3D drawing program, a patient's actual cartilage thickness is considered so that an artificial joint can be designed in an optimized form suitable for the patient's anatomical characteristics.

In order to achieve the above objective, according to an embodiment of the present disclosure, there is provided a method of reconstructing cartilage of a distal femur in which damaged cartilage of the distal femur is reconstructed to a pre-damaged state thereof for designing a patient-specific artificial knee joint, the method including: (a) generating a distal femur model A having only a bone without cartilage from image data of a femur captured by a medical imaging device; (b) generating a distal femur model B having damaged cartilage from the image data; (c) extracting a bone cross section for each region in each of the model A and the model B; (d) obtaining a distance between an articular surface of the model A and a cartilage surface of the model B at each predetermined point by using a bone cross section of the model A and a bone cross section of the model B which are extracted from the same region; and (e) generating a distal femur model C with cartilage reconstructed according to a reconstruction line offset by a thickness of the cartilage from the articular surface of the model A after presetting a specific value of the obtained distance as the thickness of the cartilage to be reconstructed.

In the embodiment of the present disclosure, the extracting (c) may include: (c-1) generating several virtual cutting surfaces relative to a specific rotational axis of a distal femur in each of the model A and the model B within a preset region of interest, and (c-2) extracting a bone cross section for each region through which each of the cutting surfaces passes in the model A and the model B by using the generated virtual cutting surfaces.

In this case, the region of interest may be preset to include a distal medial condyle and a distal lateral condyle of the distal femur, and a posterior medial condyle and a posterior lateral condyle of the distal femur.

In addition, the specific rotational axis may be a transepicondylar axis of the distal femur or a flexion-extension axis of the distal femur.

Alternatively, the specific rotational axis may be an axis passing through a medial sagittal radius center or a lateral sagittal radius center of the distal femur in a width direction of the distal femur.

In addition, in the embodiment of the present disclosure, the obtaining (d) may include: (d-1) generating combined bone cross sections by combining bone cross sections which are extracted from the same regions among a plurality of bone cross sections extracted from each of the model A and the model B, and (d-2) obtaining the distance between the articular surface of the model A and the cartilage surface of the model B at each predetermined point in each of the combined bone cross sections.

In this case, in the obtaining (d-2), a distance between the articular surface and the cartilage surface at a corresponding point may be obtained in a normal direction of the articular surface.

Meanwhile, in the embodiment of the present disclosure, in the generating (e), one of a maximum among distance values calculated from each point, a mode which occurs most frequently among distance values calculated from each point, a median among distance values calculated from each point, and an average of distance values calculated from each point may be used as the specific value.

Alternatively, in the generating (e), for each of the combined bone cross sections, the distance between the articular surface of the model A and the cartilage surface of the model B may be calculated at each predetermined point to obtain averages of the distances for the combined cross sections, and a maximum of the obtained averages of the distances for the combined cross sections may be used as the specific value.

For another example, in the generating (e), for each of the combined bone cross sections, the distance between the articular surface of the model (A) and the cartilage surface of the model (B) may be calculated at each predetermined point to extract maximums of the distances for the combined cross sections, and an average of the extracted maximums for the combined cross sections may be used as the specific value.

In order to achieve the above objective, according to another embodiment of the present disclosure, there is provided a recording medium storing a program programmed to execute a step-by-step process according to the method of reconstructing a distal femur in a computer step by step.

According to the embodiments of the present disclosure, in reconstructing the damaged cartilage of the distal femur to a pre-damaged state thereof by using a 3D drawing program, a patient's actual cartilage thickness (normal cartilage thickness and damaged cartilage thickness) is considered so that the state of the cartilage close to the pre-damaged state which is a normal state can be derived, thereby enabling the designing of an artificial joint in an optimized form suitable for the patient's anatomical characteristics.

### BRIEF DESCRIPTION OF THE DRAWINGS

The above and other objectives, features, and other advantages of the present disclosure will be more clearly understood from the following detailed description when taken in conjunction with the accompanying drawings, in which:

FIGS. 1A and 1B are views illustrating the joint line and posterior line of a distal femur used in designing an artificial joint customized for a patient;

FIG. 2 is an overall flowchart sequentially illustrating a step-by-step process for cartilage reconstruction included in a method of reconstructing cartilage of a distal femur according to an embodiment of the present disclosure;

FIG. 3 is a flowchart illustrating the detailed process of a bone cross section extraction illustrated in FIG. 2;

FIG. 4 is a flowchart illustrating the detailed process of obtaining a distance between an articular surface of a distal femur model A and the cartilage surface of a distal femur model B illustrated in FIG. 2;

FIG. 5 is a front view of the distal femur model A having only a bone without cartilage generated by a 3D drawing program on the basis of an image data of a captured femur;

FIG. 6 is a front view of the distal femur model B including damaged cartilage generated by a 3D drawing program on the basis of the image data of a captured femur;

FIGS. 7 and 8 respectively are the front view and top plan view of the distal femur for illustrating a region of interest (ROI) used at S310 of FIG. 3;

FIGS. 9A and 9B are views illustrating a state in which a virtual cutting surface is generated on the distal femur model;

FIGS. 10 and 11 are views illustrating a rotational axis which is a reference for generating the virtual cutting surface in FIGS. 9A and 9B;

FIG. 12 is a view illustrating a process of obtaining a distance d between the articular surface S1 of the model A and the cartilage surface S2 of the model B in a combined bone cross section; and

FIG. 13 is a front view of a distal femur model C in which cartilage is reconstructed on the articular surface of the model A.

### DETAILED DESCRIPTION OF THE INVENTION

Hereinafter, an exemplary embodiment of the present disclosure will be described in detail.

Prior to this, terms used in this specification are only used to describe the specific embodiment, and are not intended to limit the present disclosure. In addition, it is made clear that a singular expression may include a plural expression unless the context clearly indicates otherwise.

It should be understood that the terms “comprise”, “include”, and “have”, etc. when used in this specification, specify the presence of stated features, integers, steps, operations, elements, components, and/or combinations of them but do not preclude the presence or addition of one or more other features, integers, steps, operations, elements, components, and/or combinations thereof.

In addition, it will be understood that, although the terms first and second, etc. may be used herein to describe various elements, these elements should not be limited by these terms. These terms are only used to distinguish one element from another element.

In the description with reference to the accompanying drawings, the same reference numerals will be given to the same components, and overlapping descriptions thereof will be omitted. In addition, in describing the present disclosure, when it is determined that a detailed description of a related known technology may unnecessarily obscure the gist of the present disclosure, the detailed description thereof will be omitted.

The present disclosure is a method of reconstructing damaged cartilage of a distal femur to a pre-damaged state thereof by using a 3D drawing program for designing a patient-specific artificial knee joint (hereinafter, referred to as “an artificial joint” for convenience of explanation), wherein in reconstructing cartilage, an artificial joint can be designed in an optimized form suitable for a patient's anatomical characteristics by considering a patient's actual cartilage thickness.

The exemplary embodiment of the present disclosure will be described in detail with reference to the accompanying drawings.

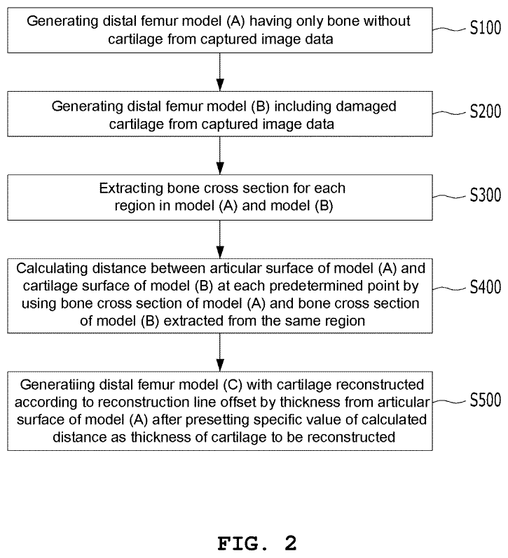

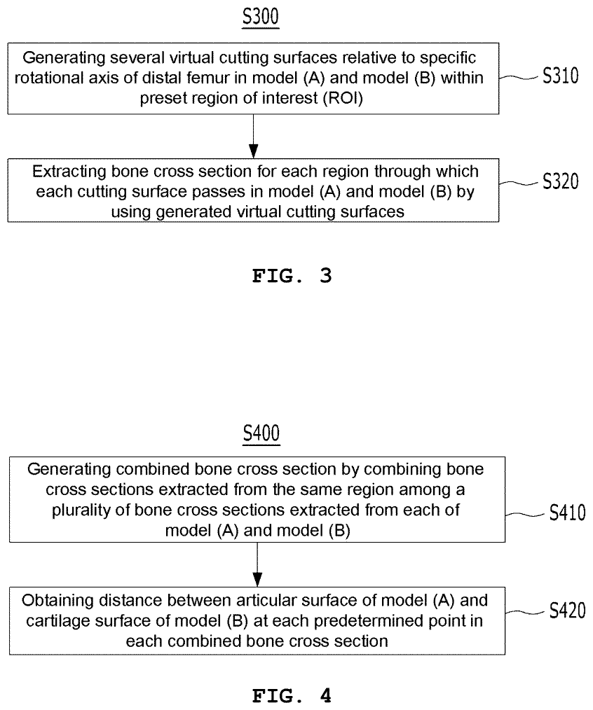

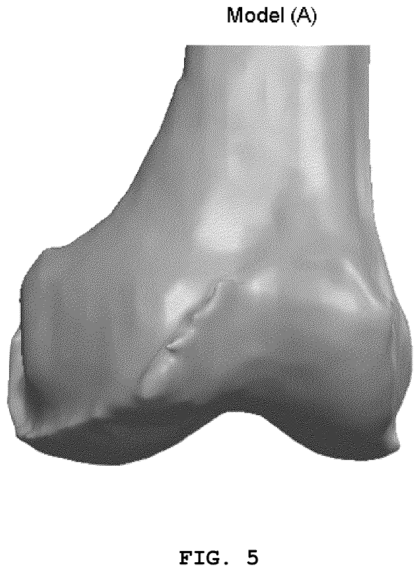

FIG. 2 is an overall flowchart sequentially illustrating a step-by-step process for cartilage reconstruction included in a method of reconstructing cartilage of a distal femur according to the embodiment of the present disclosure, FIG. 3 is a flowchart illustrating the detailed process of a bone cross section extraction illustrated in FIG. 2, and FIG. 4 is a flowchart illustrating the detailed process of S400 illustrated in FIG. 2.

Referring to FIGS. 2 to 4, the method of reconstructing cartilage of a distal femur according to the embodiment of the present disclosure may include: generating 3D models of a distal femur at S100 and S200; extracting bone cross sections from the generated 3D models at S300; obtaining a distance between an articular surface and cartilage of the distal femur by using the extracted bone cross sections at S400, and generating a distal femur model C having cartilage reconstructed by using a value of the obtained distance at S500.

Specifically, the generating of the 3D model of a distal femur may be divided into generating a distal femur model A having only a bone without cartilage from an image data of a femur captured by a medical imaging device at S100, and generating a distal femur model B including damaged cartilage from the image data of the femur captured by a medical imaging device at S200.

When generating the distal femur model A and the distal femur model B at S100 and S200, a 3D drawing program may be used. For example, an operation of generating the distal femur model A and the distal femur model B from the image data of a femur may be performed in a computer aided design/computer aided manufacturing (CAD/CAM) program environment.

At S100 and S200, the distal femur model A and the distal femur model B may be generated from the image of the femur captured by a medical imaging device by using a 3D drawing program. Here, the medical imaging device providing the image data of the femur may be a magnetic resonance imaging (MRI) device, a computed tomography (CT) device, or an X-ray device.

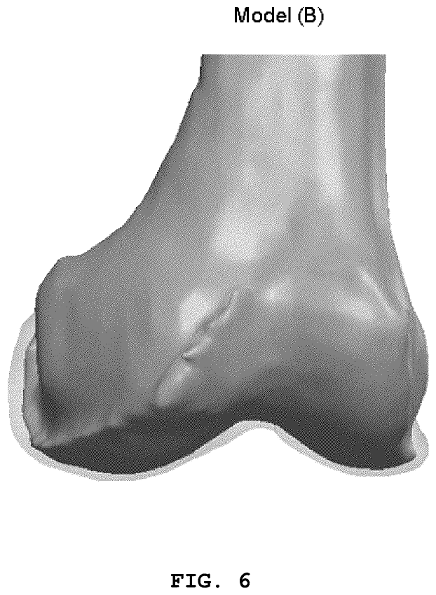

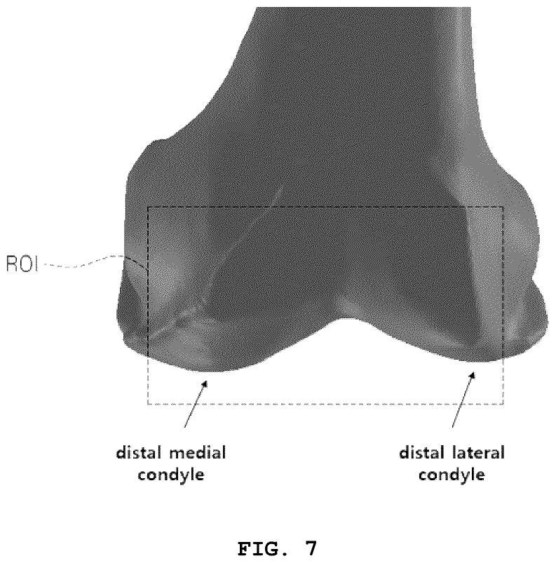

FIGS. 5 and 6 are front views of the model A and the model B generated at S100 and S200, respectively. That is, FIG. 5 is a front view of the distal femur model A having only a bone without cartilage generated by a 3D drawing program on the basis of the image data of a femur captured by a medical imaging device, and FIG. 6 is a front view of the distal femur model B including damaged cartilage generated by a 3D drawing program on the basis of the image data.

As illustrated in FIGS. 5 and 6, when the generation of the 3D models of the distal femur is completed, as a next step, extracting bone cross sections from the 3D models generated at the previous S100 and S200 are continuously performed at S300. At S300, an operation of extracting a bone cross-section for each part within the region of interest preset in each of the model A (see FIG. 5) and the model B (see FIG. 6) may be performed.

Specifically, as illustrated in FIG. 3, the extracting at S300 may be divided into generating several virtual cutting surfaces relative to a specific rotational axis of the distal femur in each of the model A and the model B within a preset region of interest (ROI) at S310, and extracting a bone cross section for each region through which each of the cutting surfaces passes in the model A and the model B by using the generated virtual cutting surfaces at S320.

The region of interest (ROI) at S310, which is a region belonging to a knee movable range in which the femur and tibia come into contact with each other when the knee is bent or extended, may be a region in which an articular cartilage is formed in the distal femur.

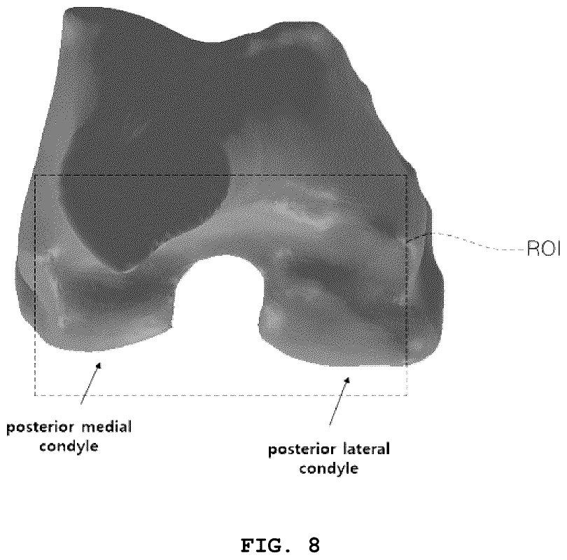

More specifically, as illustrated in the front view of FIG. 7 and the top plan view of FIG. 8, the region of interest may be preset to include the distal medial condyle and distal lateral condyle of a distal femur, and the posterior medial condyle and posterior lateral condyle of the distal femur.

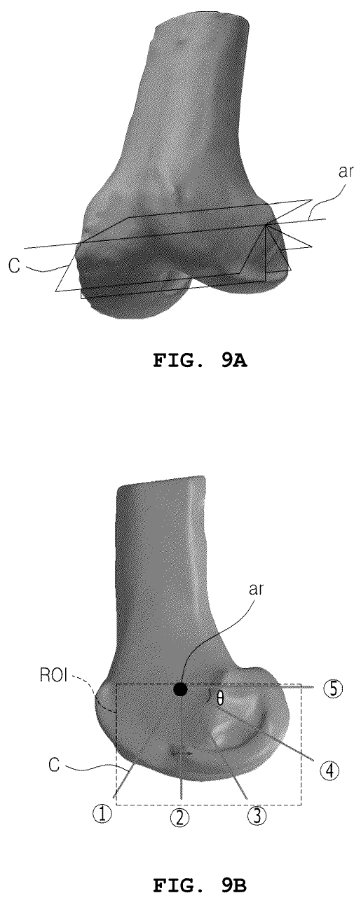

At S310, for each of the model A and the model B, several virtual cutting surfaces are created relative to the specific rotational axis ar of the distal femur. In this case, as illustrated in the perspective view of the distal femur model of FIG. 9A and the side view of FIG. 9B, the virtual cutting surfaces c are preferably generated at intervals of 0.1 to 30 degrees relative to the specific rotational axis ar in the anteroposterior direction of the distal femur within the region of interest.

When an angle θ between the virtual cutting surfaces c is less than 0.1 degree, many data (distance values between the articular surface and the cartilage surface) required for the reconstruction of cartilage can be collected, and thus thickness of the cartilage can be reconstructed to be close to a pre-damaged state thereof. However, since bone cross sections as many as the data are extracted, much information is required to be processed when combining the bone cross sections to be performed later or when extracting data from combined bone cross sections, which increases the computational load of a program.

In addition, when the angle θ between the virtual cutting surfaces c exceeds 30 degrees, the number of extracted bone cross-sections decreases, reducing a computational load in a subsequent process, but too many regions in which data are not reflected occur in the middle and cartilage is required to be reconstructed with only a small amount of data extracted from a few bone cross-sections, thereby causing a risk of inaccurate cartilage reconstruction.

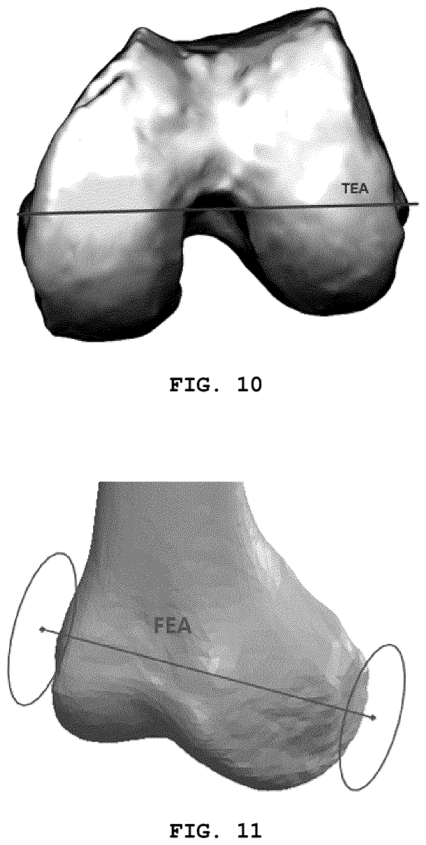

As S310, the specific rotational axis ar which is the center of the virtual cutting surfaces may be a transepicondylar axis (TEA) of the distal femur (FIG. 10) or a flexion-extension axis of the distal femur (FIG. 11), or although not shown, may be an axis passing through a medial sagittal radius center or a lateral sagittal radius center of the distal femur in the width (left and right) direction of the distal femur.

At S310, when several virtual cutting surfaces c are generated in the aforementioned manner (see FIGS. 9A and 9B), the extracting of a bone cross section for each region through which each of the cutting surfaces passes in the model A (the distal femur model having only a bone without cartilage) and the model B (the distal femur model including damaged cartilage) described above by using the generated virtual cutting surfaces c may be performed at S320.

Through S320, when a bone cross section for each region through which each of the cutting surfaces passes is extracted for each of the model A and the model B, the obtaining of a distance between the articular surface and cartilage of the distal femur may be performed by using the bone cross sections at S400. At S400, by using the bone cross section of the model A and the bone cross section of the model B which are extracted from the same region, a distance d between the articular surface of the model A and the cartilage surface of the model B is obtained at each predetermined point.

Specifically, as illustrated in FIG. 4, S400 includes generating combined bone cross sections by combining bone cross sections having the same extraction regions among a plurality of bone cross sections extracted from each of the model A and the model B at S410, and obtaining the distance d between the articular surface of the model A and the cartilage surface of the model B at each predetermined point in each of the combined bone cross sections combined through S410 at S420.

Taking a virtual cutting surface \textcircled{1} of FIG. 9B as an example, at S410, a bone cross section extracted from the model A by the virtual cutting surface \textcircled{1} and a bone cross section extracted from the model B by the same virtual cutting surface \textcircled{1} are combined with each other to generate one combined bone cross section. Of course, for bone cross sections extracted in other cutting surfaces (the virtual cutting surfaces \textcircled{2}, \textcircled{3}, \textcircled{4}, and \textcircled{5} of FIG. 9B), combined bone cross sections are generated in the same method described above.

In some cases, the model A and the model B extracted at S100 and S200 are first combined with each other, several virtual cutting surfaces are generated relative to the specific rotational axis of the distal femur in the combined model, and a combined bone cross section for each region through which each of the cutting surfaces passes in the combined model by using the generated virtual cutting surfaces is extracted. This combined bone cross section is the same as the combined bone cross section described above, and thus it should be noted that such modification may also be included in the scope of the present disclosure.

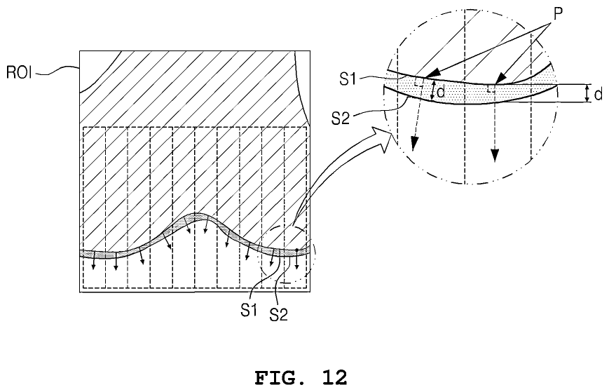

FIG. 12 is a view illustrating a combined bone cross section combining bone cross sections extracted from each of the model A and the model B by the virtual cutting surface \textcircled{2} of FIG. 9B are combined with each other. As illustrated in FIG. 12, at S420, at each predetermined point P of the combined bone cross section, a distance d between the articular surface S1 of the point and the cartilage surface S2 of the model B in a normal direction of the articular surface S1 of the model A may be obtained.

Here, the point P at which the distance d between the articular surface S1 and the cartilage surface S2 is extracted may be a point selected by a designer in a program design process. For example, as illustrated in FIG. 12, one combined bone cross section is divided into several sections in the left and right directions (parts partitioned by dotted lines), and a midpoint of each of the divided sections may be preset to be a point for extracting the distance d.

When required, in addition to the point previously determined through presetting (the midpoint of each of divided several sections as illustrated in FIG. 12), when a user directly selects a desired point by moving a cursor on a screen with an input device such as a mouse or touch pad in a state in which the combined bone cross section is displayed on the screen as illustrated in FIG. 12, the distance d may be preset to be additionally extracted for the corresponding point.

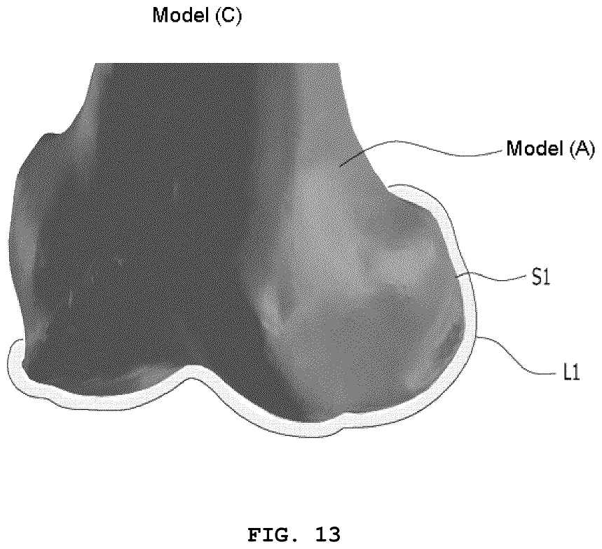

When distances (distances extracted at each point preset in combined bone cross sections) between the articular surface S1 and the cartilage surface S2 through S400 are extracted, the specific value of the distances is finally preset as thickness of cartilage to be reconstructed, and as illustrated in FIG. 13, the generating of the distal femur model C with cartilage reconstructed according to the reconstruction line L1 offset by the thickness from the articular surface S1 of the model A is performed at S500.

Specifically, at S500, after the reconstruction line L1 is formed as illustrated in FIG. 13 by offsetting by the thickness (the specific value) of cartilage to be reconstructed from the articular surface S1 in the entirety of the articular surface S1 of the model A belonging to the region of interest (ROI) described above, the distal femur model C may be generated by reconstructing the cartilage according to the reconstruction line L1.

At S500, one of a maximum of a plurality of distances calculated at predetermined points, a mode which occurs most frequently among the plurality of distances calculated at predetermined points, a median of the plurality of distances calculated at predetermined points, and an average of the plurality of distances calculated at predetermined points may be used as the specific value.

Preferably, one of a maximum of a plurality of distances calculated from each of several combined bone cross sections generated at S410, a mode which occurs most frequently among the plurality of calculated distances, a median of the plurality of calculated distances, and an average of the plurality of calculated distances may be used as the specific value.

In some cases, in each of the combined bone cross sections generated through S410, the distance d between the articular surface of the model A and the cartilage surface of the model B is calculated at each predetermined point to obtain averages of the distances for the combined bone cross sections, and a maximum of the obtained averages of the distances for the combined bone cross sections may be used as the specific value.

For example, when the averages of the distances d of five combined bone cross sections extracted and combined by the virtual cutting surfaces \textcircled{1} to \textcircled{5} illustrated in FIG. 9B are 1 mm, 1.2 mm, 1.1 mm, 1.5 mm, and 2 mm, respectively, 2 mm which is the maximum of the averages of the distances is selected as the specific value, and thus in the entirety of the articular surface S1 of the model A belonging to the region of interest (ROI), the reconstruction line L1 offset by 2 mm from the articular surface S1 is formed.

As another preferred example, in each of the several combined bone cross sections generated through S410, the distance between the articular surface of the model A and the cartilage surface of the model B is calculated at each predetermined point to extract the maximums of the distances for the combined bone cross sections, and an average of the extracted maximums for the combined bone cross sections may be considered to be used as the specific value.

In this case, when maximums of a plurality of distances extracted from each of five combined bone cross sections extracted and combined by the virtual cutting surfaces \textcircled{1} to \textcircled{5} illustrated in FIG. 9B are 1 mm, 1.2 mm, 1.1 mm, 1.5 mm, and 2 mm, respectively, 1.36 mm which is the average of the maximums is selected as the specific value, and thus in the entirety of the articular surface S1 of the model A belonging to the region of interest (ROI), the reconstruction line L1 offset by 1.36 mm from the articular surface S1 is formed.

Even in the reconstructing of cartilage at S500, in addition to the method of forming the reconstruction line L1 by applying the specific value to the entirety of the articular surface S1 of the model A belonging to the region of interest (ROI), a method of reconstructing cartilage on the articular surface of the model A by applying a different reconstruction line L1 to each section divided by the virtual cutting surfaces described above may also be considered.

In other words, cartilage may be reconstructed in such a manner that the articular surface of the model A is subdivided into an articular surface section between the cutting surfaces \textcircled{1} and \textcircled{2}, an articular surface section between the cutting surfaces \textcircled{2} and \textcircled{3}, an articular surface section between the cutting surfaces \textcircled{3} and \textcircled{4}, and an articular surface section between the cutting surfaces \textcircled{4} and \textcircled{5} in FIG. 9B, and after calculating a specific value for each of the subdivided sections, the calculated specific value is applied to a corresponding section.

For example, in a combined bone cross section generated by the cutting surface \textcircled{1}, the distances d are calculated at predetermined points to extract the maximum of the distances, and in a combined bone cross section generated by the cutting surface \textcircled{2}, the distances d are calculated at predetermined points to extract the maximum of the distances, and then the average of the distance maximum in the cutting surface \textcircled{1} and the distance maximum in the cutting surface \textcircled{2} is obtained, and this average may be used as the specific value in the articular surface section between the cutting surface \textcircled{1} and the cutting surface \textcircled{2}.

Even in the articular surface section between the cutting surfaces \textcircled{2} and \textcircled{3}, the articular surface section between the cutting surfaces \textcircled{3} and \textcircled{4}, and the articular surface section between the cutting surfaces \textcircled{4} and \textcircled{5}, each of specific values is extracted in the same manner and applied to a corresponding section, so that cartilage can be reconstructed by being divided into sections.

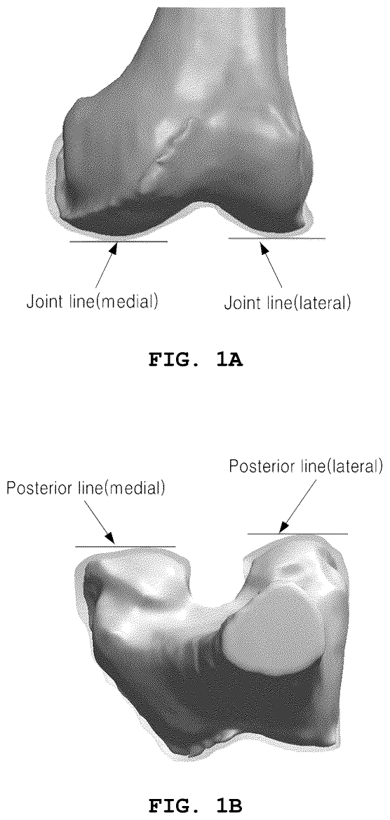

Meanwhile, when the distal femur model C with cartilage reconstructed through S500 is generated, joint lines and posterior lines (see FIG. 1) mentioned in the background art are generated from the model C, and when an artificial joint is designed by considering height difference between the generated joint lines and height difference between the posterior lines, the artificial joint can be designed in an optimal form that fits a patient's actual anatomical characteristics.

The method of reconstructing cartilage of a distal femur according to the embodiment of the present disclosure described above may be applied only to a lateral side when a subject has a varus leg to derive the thickness of the cartilage, or may be applied only to a medial side when a subject has a valgus leg to derive the thickness of the cartilage.

A conventional technology adopts a method in which a CT image of the distal femur is used to reconstruct the bone of the distal femur in the form of a 3D digital model by using a 3D drawing program, a virtual cartilage is generated by being offset by a predetermined distance from the articular surface of the reconstructed 3D bone model, and a joint line and a posterior line are generated from the generated virtual cartilage as illustrated in FIG. 1 so that the lines are considered to design an artificial joint.

However, in this method, the virtual cartilage is generated by being offset by a predetermined thickness (approximately 1 to 2 mm) from the articular surface of the 3D bone model without considering the thickness of an actual cartilage that is not damaged, and accordingly, the thickness of the generated virtual cartilage is smaller or greater than the thickness of the normal cartilage before damage. Therefore, there is a problem in that the artificial joint cannot be designed in an optimal form in which the artificial joint fits a patient's actual anatomical characteristics.

On the other hand, according to the embodiment of the present disclosure, in reconstructing the damaged cartilage of a distal femur to a pre-damaged state thereof by using a 3D drawing program, a patient's actual cartilage thickness (normal cartilage thickness and damaged cartilage thickness) is considered so that the state of the cartilage close to the pre-damaged state which is a normal state can be derived, thereby enabling the designing of an artificial joint in an optimized form suitable for the patient's anatomical characteristics.

In the above detailed description of the present disclosure, only the specific embodiment has been described. However, it should be understood that the present disclosure is not limited to the particular form mentioned in the detailed description, but rather includes all modifications, equivalents, and alternatives within the spirit and scope of the present disclosure as defined by the appended claims.

What is claimed is:

1. A method of reconstructing cartilage of a distal femur in which damaged cartilage of the distal femur is reconstructed to a pre-damaged state thereof for designing a patient-specific artificial knee joint, the method comprising:
(a) generating a distal femur model (A) having only a bone without cartilage from image data of a femur captured by a medical imaging device;
(b) generating a distal femur model (B) having damaged cartilage from the image data;
(c) extracting a bone cross section for each region in each of the model (A) and the model (B);
(d) obtaining a distance (d) between an articular surface of the model (A) and a cartilage surface of the model (B) at each predetermined point by using a bone cross section of the model (A) and a bone cross section of the model (B) which are extracted from the same region; and
(e) generating a distal femur model (C) with cartilage reconstructed according to a reconstruction line (L1) offset by a thickness of the cartilage from the articular surface of the model (A) after presetting a specific value of the distance (d) obtained in the obtaining (d) as the thickness of the cartilage to be reconstructed.

2. The method of claim 1, wherein the extracting (c) comprises:
(c-1) generating several virtual cutting surfaces relative to a specific rotational axis of a distal femur in each of the model (A) and the model (B) within a preset region of interest, and
(c-2) extracting a bone cross section for each region through which each of the cutting surfaces passes in the model (A) and the model (B) by using the generated virtual cutting surfaces.

3. The method of claim 2, wherein the region of interest comprises a distal medial condyle and a distal lateral condyle of the distal femur, and a posterior medial condyle and a posterior lateral condyle thereof.

4. The method of claim 2, wherein the specific rotational axis is a transepicondylar axis of the distal femur or a flexion-extension axis of the distal femur.

5. The method of claim 2, wherein the specific rotational axis is an axis passing through a medial sagittal radius center or a lateral sagittal radius center of the distal femur in a width direction of the distal femur.

6. The method of claim 2, wherein the obtaining (d) comprises:
(d-1) generating combined bone cross sections by combining bone cross sections which are extracted from the same regions among a plurality of bone cross sections extracted from each of the model (A) and the model (B), and
(d-2) obtaining the distance between the articular surface of the model (A) and the cartilage surface of the model (B) at each predetermined point in each of the combined bone cross sections.

7. The method of claim 6, wherein in the obtaining (d-2), the distance between the articular surface and the cartilage surface at the predetermined point is obtained in a normal direction of the articular surface.

8. The method of claim 6, wherein in the generating (e), one of a maximum among distance values calculated from each point, a mode which occurs most frequently among distance values calculated from each point, a median among distance values calculated from each point, and an average of distance values calculated from each point is used as the specific value.

9. The method of claim 6, wherein in the generating (e), for each of the combined bone cross sections, the distance between the articular surface of the model (A) and the cartilage surface of the model (B) is calculated at each predetermined point to obtain averages of the distances for the combined cross sections, and a maximum of the obtained averages of the distances for the combined cross sections is used as the specific value.

10. The method of claim 6, wherein in the generating (e), for each of the combined bone cross sections, the distance between the articular surface of the model (A) and the cartilage surface of the model (B) is calculated at each predetermined point to extract maximums of the distances for the combined cross sections, and an average of the extracted maximums for the combined cross sections is used as the specific value.

11. The method of claim 6, wherein in the generating (e), the specific value is applied to an entirety of the articular surface S1 of the model (A) belonging to the region of interest (ROI) to form the reconstruction line (L1).

12. The method of claim 6, wherein in the generating (e), the specific value for each section divided by the virtual cutting surfaces is used to form the reconstruction line (L1) for the each section.

13. A non-transitory recording medium storing a program programmed to execute a step-by-step process according to the method of reconstructing a distal femur according to claim 1 in a computer step by step.
